(19) United States
(12) Patent Application Publication
(10) Pub. No.: US 2022/0347322 A1
(43) Pub. Date: Nov. 3, 2022

(54) METHOD OF TREATING ARTERIOVENOUS MALFORMATIONS BY TARGETING THE EPHRIN PATHWAY

(71) Applicant: THE REGENTS OF THE UNIVERSITY OF CALIFORNIA, Oakland, CA (US)

(72) Inventor: Rong Wang, San Francisco, CA (US)

(21) Appl. No.: 17/764,892

(22) PCT Filed: Sep. 30, 2020
(86) PCT No.: PCT/US20/53566

§ 371 Date: Mar. 29, 2022

Related U.S. Application Data
(60) Provisional application No. 62/908,525, filed on Sep. 30, 2019.

(51) Int. Cl. A61K 49/00; G01N 33/50

(52) U.S. Cl. CPC A61K 49/0008; G01N 33/5041; G01N 2800/7033

(57) ABSTRACT

The disclosure provides a mouse model of arteriovenous malformation, such as found in Hereditary Hemorrhagic Telangiectasia, that accurately and persistently models the disease progression in various organisms, including humans. The disclosure further provides a mouse comprising a mutant Ephrin pathway gene, such as Alk1, in brain endothelial cells only, and methods of screening for therapeutically useful modulators of Ephrin pathway gene expression or gene product activity useful in treating or ameliorating a symptom of arteriovenous malformation, such as Hereditary Hemorrhagic Telangiectasia or hemorrhagic stroke.

### CROSS-REFERENCE TO RELATED APPLICATIONS

This application claims the priority benefit under 35 U.S.C. § 119(e) of U.S. Provisional Patent Application No. 62/908,525, filed Sep. 30, 2019, which is incorporated herein by reference in its entirety.

### STATEMENT OF GOVERNMENT SUPPORT

This invention was made with government support under grant no. NS067420 awarded by The National Institutes of Health. The government has certain rights in the invention.

### FIELD

The disclosure relates to the fields of medicine and genetics.

### BACKGROUND

Hereditary Hemorrhagic Telangiectasia (HHT), or Osler-Weber-Rendu disease, is an autosomal dominant disorder with high penetrance that is characterized by malformed blood vessels, particularly multifocal arteriovenous malformations (AVMs). Arteriovenous (AV) malformations (AVMs) are abnormal shunts from arteries directly into veins, displacing capillaries required for blood perfusion (FIG. 1). Telangiectases are small AVMs, connecting dilated arterioles directly to dilated venules, without capillary involvement. AVMs can rupture, leading to ischemia, hemorrhage, and impaired organ function. AVMs can occur anywhere in the body, but brain AVMs (BAVMs) are the most dangerous. BAVMs can cause seizures, severe headaches, vertigo, vision loss, intracranial bleeding, neurological dysfunction, stroke, and even death. High-pressure blood flow strains the dilated venules or veins, resulting in frequent rupture and recurrent heavy bleeding. HHT patients present with hemorrhage-prone AVMs in the lungs, liver, brain, skin, and other organs, resulting in catastrophic complications. Systemic AV shunting can also lead to high-output cardiac failure and death.

HHT remains a serious condition that is difficult to treat, and there are no good treatment options for HHT patients. HHT cannot yet be prevented or cured. Clinical trials showed that anti-VEGF treatment was not effective in reducing or eliminating most HHT symptoms, except for a slight reduction of nosebleed with nasal spray of the composition.

ALK1 loss-of-function mutations are responsible for type 2 HHT (HHT2) cases. Alk1 encodes a type I receptor for transforming growth factor 6 (TGFβ) superfamily ligands, including bone morphogenetic proteins (BMPs). The Alk1 receptor is predominantly expressed in arterial endothelial cells (ECs) (FIG. 3). The TGFβ superfamily ligands each bind to a specific endothelial cell (EC) surface receptor complex composed of Alk1 and a type II receptor, which then phosphorylates Alk1. Phosphorylated Smad1/5/8 complexes then bind to Smad4, which translocates to the nucleus to transcriptionally regulate target genes. Collectively, the heterodimeric complexes determines ligand specificity to control many processes in development, and in disease (FIG. 2).

Additional events beyond germline mutation of ALK1 are critical in HHT pathogenesis. In most cases, a localized, somatic mutation of the other normal allele is thought to result in loss of heterozygosity (LOH). Most human ALK1 mutations are missense or nonsense mutations, implying that haploinsufficiency of the Alk1 protein underlies HHT1. In adult mouse, however, homozygous loss of Alk1 is necessary, but not sufficient, to initiate AVMs. An environmental event, such as angiogenesis, inflammation, or injury, is also required for AVM formation in mice. Identification of such events in vivo has been hindered by the lack of a robust animal model. Existing animal models with one or both copies of ALK1 deleted are limited in various ways. Heterozygous germline Alk1 knockout mice develop mild lesions with long latency (7-18 months) and incomplete penetration. Deletion of Alk1 alleles in ECs throughout the body in neonates using Cdh5(PAC)CreER^{T2} leads to AVM-like phenotypes in the retina. However, these mice die within 4 days of Alk1 deletion, which is too soon to allow BAVM development.

HHT remains a serious condition that is difficult to treat, and there are no good treatment options for HHT patients. HHT cannot yet be prevented or cured. Identification of drug targets and candidate drugs has been hindered by the lack of a robust animal model. Existing animal models are limited in various ways. Heterozygous germline Alk1 knockout mice develop mild lesions with long latency (7-18 months) and incomplete penetration. Deletion of Alk1 alleles in endothelial cells throughout the body in neonates leads to AVM-like phenotypes in the retina. However, these mice die within 4 days of Alk1 deletion, too soon to allow Brain AVM development.

HHT is a devastating inherited condition with high penetrance from a young age. No prevention or cure exists for the major clinical manifestations of HHT, highlighting a need in the art for better treatment strategies and methodologies. In addition and more specifically, a better preclinical animal model that faithfully reflects BAVM presentation in HHT2 patients is critically needed to understand disease progression and develop preventative or treatment strategies.

HHT can cause bleeding in several different organs of the body. People with HHT live with recurring nosebleeds. Nosebleeds in people with HHT can vary in severity from a simple nuisance to bleeds that require blood transfusion. Other commonly affected organs are the brain, lungs, and GI tract.

Thus, a need continues to exist in the art for methods of preventing and/or treating arteriovascular malformations, such as the AVMs characteristic of a number of vascular malformation diseases, including hereditary hemorrhagic telangiectasia and hemorrhagic stroke.

### SUMMARY

Based on the data disclosed herein, a preclinical HHT2-BAVM mouse model has been developed to identify molecular regulators crucial for AVM pathogenesis. As disclosed herein, the model incorporates both a targeted approach and unbiased genome-wide expression profiling to elucidate the molecular interactions involved in AVM formation. The model disclosed herein faithfully reflects disease presentation in HHT2 patients, in contrast to current models of this disease. This mouse model of HHT2-BAVM involves a deletion of both Alk1 alleles specifically in brain endothelial cells (ECs), i.e., brain ECs, and only brain ECs, have homozygous deletions of Alk1. The data show that this deletion results in robust BAVM, intracranial hemorrhages, and neurological consequences, without detectable defects elsewhere in the body.

Disclosed herein are methods for preventing or treating arteriovenous malformations (AVMs) and methods for preventing or treating Hereditary Hemorrhagic Telangiectasia (HHT) by administering a therapeutically effective amount of a modulator of the Eph receptor/ephrin pathway. In exemplary methods, arteriovenous malformations in HHT are prevented or treated, arteriovenous malformations not associated with HHT are prevented or treated, and HHT not associated with an AVM are prevented or treated. For example, the methods of the disclosure are useful in preventing or treating hemorrhagic stroke, which may or may not be associated with HHT. Further, AVMs being prevented or treated according to the methods disclosed herein may be brain AVMs (BAVMs) or AVMs found outside the brain.

The methods disclosed herein were developed using brain AVM as a model to develop treatment for HHT. AVMs can occur in other parts of the body, including the lung, nose, skin, and the like, with similar underlying cause. The methods disclosed herein, developed with brain AVMs, are expected to apply to the treatment of HHT lesions in other parts of the body.

Beyond HHT mutation-induced brain AVM, brain AVMs also occur without HHT-mutations. Whether HHT-induced or not, current treatments for brain AVMs include costly, risky neurosurgical resection, endovascular embolization, and neuroradiotherapy, which may be more detrimental to some patients than no treatment at all. The methods disclosed herein, developed with brain AVMs, are expected to apply to treatment beyond HHT patients, i.e., patients with AVMs but not HHT.

“Preventing,” as used herein, encompasses methods that achieve one or more of the following: preventing the onset of a vascular malformation conditions; or delaying or halting the progression of a vascular malformation condition.

“Treating,” as used herein, refers to a method that reduces or delays the magnitude or appearance of a symptom characteristic of a vascular malformation condition, or slows the progression of a vascular malformation condition.

In some embodiments “treatment,” as used herein, means achieving one or more physiological, physical, functional, therapeutic, or performance outcomes. For example, treatment may encompass: alternative Eph receptor/ephrin (i.e., Eph receptor and/or ephrin) signaling in one or more blood vessels; and/or improving vascular functions and circulations of one or more blood vessels.

The treatments of the invention may achieve local effects, for example, treating vascular malformation at a site or in an organ, or may achieve systemic effects, for example, preventing bleed and improving circulation generally throughout the body.

In one aspect, the disclosure provides a method of treating arteriovenous malformation in a subject comprising administering a therapeutically effective amount of a modulator of Eph receptor/ephrinB2 signaling to the subject. In some embodiments, the modulator inhibits Eph receptor/ephrin B2 signaling. In some embodiments, the modulator stimulates Eph receptor/ephrin B2 signaling. In some embodiments, the subject has hereditary hemorrhagic telangiectasia. In some embodiments, the subject is at risk of, or has had, a hemorrhagic stroke. In some embodiments, the arteriovenous malformation is in the brain.

Another aspect of the disclosure is a method of treating hereditary hemorrhagic telangiectasia comprising administering a therapeutically effective amount of a modulator of an Eph receptor polypeptide. In some embodiments, the modulator is an inhibitor of the Eph receptor. In some embodiments, the Eph receptor is an EphA receptor, such as EphA1, EphA2, EphA3, EphA4, EphA5, EphA6, EphA7, EphA8, EphA9, or EphA10. In some embodiments, the Eph receptor is an EphB receptor, such as EphB1, EphB2, EphB3, EphB4, EphB5 or EphB6. In some embodiments, the Eph receptor is an Eph type-B receptor, such as Eph type-B receptor 4 (EphB4). In some embodiments, the inhibitor is soluble Eph type-B receptor 4. In some embodiments, the Eph type-B receptor is Eph type-B receptor 1 (EphB1), Eph type-B receptor 2 (EphB2), Eph type-B receptor 3 (EphB3), or Eph type-B receptor 6 (EphB6).

Another aspect of the disclosure is drawn to a method of treating hereditary hemorrhagic telangiectasia comprising administering a therapeutically effective amount of a modulator of an ephrin polypeptide. In some embodiments, the modulator is a stimulator of the ephrin polypeptide. In some embodiments, the ephrin polypeptide is an ephrin type-A polypeptide, such as ephrin A1, ephrin A2, ephrin A3, ephrin A4, ephrin A5 or ephrin A6. In some embodiments, the ephrin polypeptide is an ephrin type-B polypeptide, such as ephrin B2. In some embodiments, the ephrin type-B polypeptide is ephrin 131 or ephrin B3.

Another aspect of the disclosure provides a method of screening for a therapeutic to treat hereditary hemorrhagic telangiectasia comprising (a) administering a candidate compound to an organism with a brain-specific arteriovenous malformation (BAVM organism); (b) maintaining the organism for a time suitable for AVM symptoms to arise; (c) measuring a property associated with brain arteriovenous malformation (BAVM) in a BAVM organism; and (d) comparing the level of the property in the BAVM organism receiving the candidate compound to the level of the property in a BAVM organism not receiving the candidate compound, wherein a candidate compound is identified as a therapeutic to treat hereditary hemorrhagic telangiectasia if the level of the property differs in the BAVM organism receiving the candidate compound relative to the level of the property in the BAVM organism not receiving the candidate compound. In some embodiments, the BAVM organism is a mouse. In some embodiments, the property is a symptom of BAVM, such as intracranial bleeding. In some embodiments, the symptom is hemorrhagic stroke. In some embodiments, the property is AVM onset, AVM size, extent of hemorrhage, or time to moribundity.

Yet another aspect of the disclosure is an in vitro method of screening for a therapeutic to treat hereditary hemorrhagic telangiectasia comprising (a) administering a candidate compound to an alk1^{-/-} endothelial cell derived from a brain; (b) measuring the level of an arteriovenous programming protein; and (c) comparing the level of the arteriovenous programming protein in the presence of the candidate compound to the level of the arteriovenous programming protein in the absence of the compound, wherein a candidate compound is identified as a therapeutic to treat hereditary hemorrhagic telangiectasia if the level of an arteriovenous programming protein is differs in the presence of the candidate compound compared to the level in the absence of the candidate compound. In some embodiments, the arteriovenous programming protein is a protein in the Ephrin pathway. In some embodiments, the protein in the Ephrin pathway is an ephrin type-B or an Eph type-B receptor. In some embodiments, the protein in the Ephrin pathway is Eph type-B receptor 4 or ephrin B2. In some embodiments, the arteriovenous programming protein is a protein in the Notch pathway or the Transforming Growth Factor-13 pathway.

Still another aspect of the disclosure provides a non-human mammal comprising a homozygous Alk1⁻ inactivating mutation exclusively in brain endothelial cells. In some embodiments, the mammal is a mouse. In some embodiments, the homozygous Mkt inactivating mutation is a deletion of Alk1.

Another aspect of the disclosure is a method of making the non-human mammal disclosed herein comprising the use of Crispr/Cas9 to introduce the mutation. In some embodiments, the method further comprises determining that a brain endothelial cell harbors the mutation by single-cell sequencing.

The disclosure comprehends modulation of Eph receptor/ephrin signaling in blood vessels, wherein the expression or activity of one or more members of the pathways (16 Eph receptors and 9 ephrin ligands) are modulated, as well as modulators of the signaling.

Modulation of Eph receptor/ephrin signaling includes modifying Eph receptor/ephrin signaling by increasing or decreasing Eph receptor/ephrin activity, upregulating or downregulating Eph receptor/ephrin expression or activity, and/or activating or inhibiting one or more of the Eph receptors or ephrin ligands, in one or more vessels.

The disclosure comprehends the administration of Modulators of Eph receptor/ephrin signaling to prevent and/or treat a variety of vascular malformation conditions, including HHT and hemorrhagic stroke. The modulator of Eph receptor/ephrin signaling comprises any composition of matter that changes the signaling in cells of the body, for example, blood vessel endothelial cells, for example arterial endothelial cells, and/or venous endothelial cells, and/or capillary endothelial cells. The modulator of Eph receptor/ephrin signaling may comprise any agent having Eph receptor/ephrin signal-inhibiting or signal-activating activity, including, for example, antibodies, small molecules, peptides and proteins, nucleic acids, RNAs, plant extracts, and herbal medicines. In one embodiment, the modulator of Eph receptor/ephrin signaling is a small molecule.

A modulator of the Notch pathway may be a peptide or protein activator of a Notch pathway protein.

The disclosure also contemplates modulators of an Eph receptor/ephrin that are partial or full-length Eph receptor or ephrin ligand, as well as sequence variants (no more than 10 amino acid changes, and preferably no more than three amino acid changes) and mimics of the wild-type Eph receptor or ephrin. Variants may also comprise truncations of the wild type proteins.

In some embodiments, the peptide or protein modulator of Eph receptor/ephrin signaling is a mimic, including engineered variants and de novo synthetic molecules comprising amino acid sequences, peptides, and proteins that are capable of modifying Eph receptor/ephrin signaling. For example, the engineered variants may comprise ligand binding domains of a receptor and other active domains thereof, for example, altered to have modifying activity for Eph receptor/ephrin signaling.

In some embodiments, the modulator of Eph receptor/ephrin signaling comprises an antibody, or antigen binding fragment thereof, wherein the antibody binds to a receptor or a ligand. For example, antibodies against EphB4 or ephrinB2. The disclosure comprehends an antibody that is an inhibiting antibody or an activating antibody.

In some embodiments, the modulator of Eph receptor/ephrin signaling comprises a lipid.

In some embodiments, the modulator of Eph receptor/ephrin signaling is a nucleic acid, for example a genetic construct that is delivered to target cells and expressed by such cells. In some embodiments, the modulator that is a Notch-activating agent is a nucleic acid construct that codes an Eph receptor or ephrin ligand, full or partial protein. In some embodiments, the construct encodes an EphB4 receptor. In some embodiments, the construct encodes an Eph receptor or an ephrin ligand extracellular domain, for example. In some embodiments, the construct encodes an Eph receptor or an ephrin ligand intracellular domain, for example.

The genetic construct that is a modulator of Eph receptor/ephrin signaling may comprise an expression vector of any type including, for example, a gene construct delivered by gene therapy technologies. Exemplary vectors and technologies include a viral vector (e.g., adenovirus or adeno-associated virus, lentivirus), clustered regularly interspaced short palindromic repeats-associated nuclease system (CRISPR/Cas) type constructs, CRISPRa, CRISPRi, nanoparticle mediated gene delivery (e.g., dendrimers, lipids, chitosan gene delivery particles, and the like) or any other gene therapy constructs known in the art. The genetic construct may further comprise a constitutive promoter for high levels of expression or an inducible promoter for controlled expression of a modulator of Eph receptor/ephrin signaling. The promoter may be a tissue-specific promoter, for example, an endothelial-specific promotor, VE-cadherin promoter, for example, an arterial-specific promoter BMX, DLL4, Notch4, connexin 40, connexin 43, connexin 37 or, for example, the venous-specific promoter APJ, and for example a capillary-specific promotor.

In one embodiment, the modulator of Eph receptor/ephrin signaling is an RNA that affects Eph receptor/ephrin signaling, such as a microRNA, RNAi construct, short hairpin RNA or other RNA sequence that can increase or decrease Eph receptor/ephrin signaling. For example, the RNA may comprise a micro-RNA or other RNA that inhibits expression or activity of Eph receptor/ephrin signaling. The RNA construct may comprise a transient expression vector for the expression of a modulator of Eph receptor/ephrin signaling.

Nucleic acid construct delivery to target cells, for example, endothelial cells of the vessel, may be achieved by any means known in the art. For example, delivery may be achieved by viral gene vectors, electroporation, biolistic delivery systems, microinjection, ultrasound, hydrodynamic delivery, liposomal delivery, polymeric or protein-based cationic agents (e.g., polyethylene imine, polylysine), intraject systems, and DNA-delivery dendrimers. Gene delivery may be systemic (e.g., intravenous), or localized, for example, by localized injection, delivery by catheters, such as drug-eluting balloon catheters, or by drug-eluting implants, such as stents. Liposomal delivery systems may also be used, for example, in methods of delivering transgenes to be expressed in vascular tissues. Methods for targeted delivery to blood vessels may be adapted from methods known in the art.

Other features and advantages of the disclosure will be better understood by reference to the following detailed description, including the drawing and the examples.

### BRIEF DESCRIPTION OF THE DRAWINGS

FIG. 1. Arteriovenous Malformation. (A) In the normal vascular bed arteries are connected to veins through capillaries. (B) Arteriovenous malformation is an enlarged connection between the artery and the vein, which exhibits decreased resistance and increased blood flow.

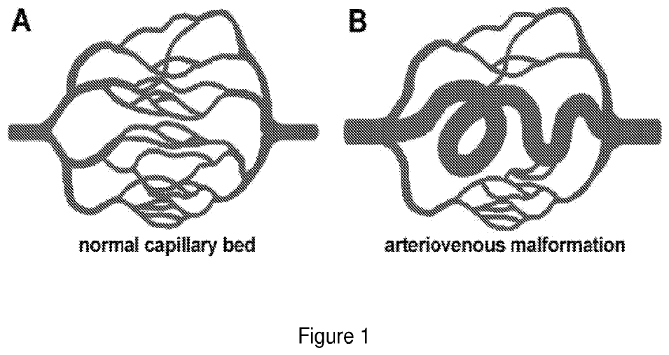

FIG. 2. BMP signaling pathway and Hereditary Hemorrhagic Telangiectasia (HHT).

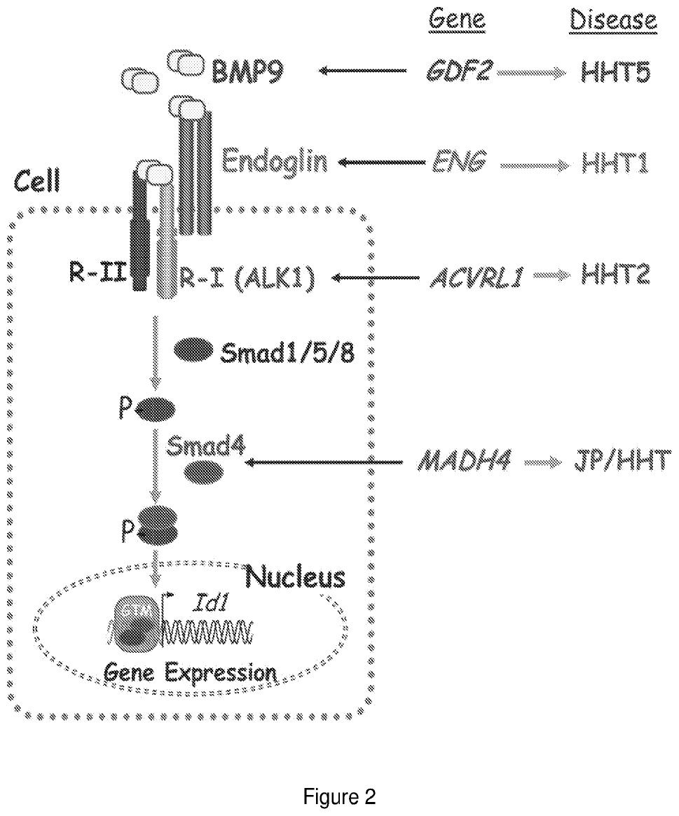

FIG. 3. Alk1 is expressed in arteries. Whole-mount X-gal (5-bromo-4-chloro-3-indolyl-13-D-galactopyranoside) staining of a brain with an Alk1-LacZ reporter gene to detect Alk1 transcription. (Scale bar, 500 μm.)

FIG. 4. 5D 2P live image through a cranial window. 5D 2P live image through a cranial window deep into the cortex for 3D structure at single-cell resolution, along with blood velocity measurement over days.

FIG. 5. Moribundity curve in Slco1c1-CreER^{T2}; Alk1^{fx/fx} mice. Tamoxifen (TAM) was a injected at post-natal day 13 (P13), and all mutants died by P22.

FIG. 6. Hemorrhages in Slc1c1-CreER^{T2}; Alk1^{fx/fx} brains. Cerebral hemorrhages, similar to human HHT, in all mutants. B, P20, CD, P26. TAM injection at P13. White scale bar, 5 mm; black scale bar, 0.5 mm.

FIG. 7. Seizure and ataxia in SlcCreER^{T2}; Alk1^{fx/fx} mice. Still frames were taken from a movie of affected mice (green arrows).

FIG. 8. Microfil Casting of Slc1c1-CreER^{T2}; Alk1^{fxf/x} brains. Multifocal AVMs (red asterisks), resembling human HHT, developed in all mutants. Scale bars: white, 5 mm; red, 0.5 mm.

FIG. 9. Time-lapse 2-Photon imaging. Time-lapse 2-Photon imaging through a cranial window show Slco1c1-CreER^{T2}; Alk1^{fx/fx} mice developed AV shunts through enlargement of capillary-like vessels, similar to human HHT. (Scale bars, 50 μm.)

FIG. 10. Ephrin-B2H2B-eGFP expression on brain arterial ECs. (A & B) In vivo time-lapse imaging shows brain vessels labeled by TRITC-dextran. Nucleus eGFP represents arterial ECs. (C) R26R-RG fluorescent reporter labels cells with red nuclei and green cytoplasm after Cre recombination.

FIG. 11. Tracking endothelial cells. Endothelial cells were tracked in brain AV shunt development using two-photon imaging in live mice.

FIG. 12. EphB4 expression by LacZ reporter. Whole-mount β-gal (β-galactosidase) staining at P16, showing increased expression in veins (v), AV connections, but not in arteries with an Alk1 deletion.

FIG. 13. Heterozygous deletion of EphB4. Heterozygous deletion of EphB4 reduced hemorrhage in Alk1 deletion at P20. TAM administered at P13. (Scale bar, 5 mm.)

FIG. 14. Moribundity. Heterozygous deletion of EPHB4 extends moribund time of Alk1 deletion. TAM was administered at P13.

FIG. 15. Heterozygous deletion of Ephb4 attenuated AV shunting formation. In Slco1c1-CreER^{T2}; Alk1^{flox/flox}, enlarged AV connections directly connect arteries (A) to veins (V). Heterozygous deletion of EPHB4 (Slco1c1-CreER^{T2}; Alk1^{flox/flox}, EPHB4^{LacZ/+}) reduced diameters of AV connections. Diameters of AV connections from 5 pairs have been quantified by imageJ.

### DETAILED DESCRIPTION

Alk1 is predominantly expressed in arterial endothelial cells (ECs), making it an excellent candidate to investigate the role of AV-specific genes in AVM pathogenesis. Disclosed herein is the deletion of Alk1 specifically from brain ECs, avoiding lethal complications of whole-body Alk1 deletion, using a recently characterized Slco1c1-CreER^{T2} allele [Slco1c1-CreER^{T2} mice] and another mouse line, i.e., the Alk1^{flox/flox} mouse line, a temporally inducible Cre under the control of the Slco1c1 promoter (active specifically in the brain endothelium). Deletion of A/kl by Tamoxifen (TAM) in this Slco1c1-CreER^{T2}; Alk1^{fx/fx} model (the HHT2 mouse model as referenced herein) led to Alk1 deletions specific to brain ECs, avoiding the lethal complications of whole-body Alk1 deletions. The result was 100% BAVM and intracranial hemorrhages, without detectable defects elsewhere in the body. Using this model, the HHT phenotype was fully characterized within one month, establishing the model disclosed herein as a robust mouse model of HHT.

The HHT2 mouse model disclosed herein has been characterized using innovative, high-resolution two-photon imaging through a cranial window to access the vasculature in live brains, achieving a 5D perspective (3D vascular structure plus blood velocity over time). The model also provides the tools for screening candidate modulators (e.g., molecular regulators) that promote or hinder HHT2 BAVM formation. In addition, cutting-edge genomic expression profiling is used to elucidate Alk1 target genes.

The HHT2-BAVM mouse model with brain endothelial cell-specific Alk1 mutations (e.g., deletions) is expected to reveal the hallmarks of BAVM manifestations, including gross pathology, histopathology, hypoxia, perfusion, vessel densities, patterns, and AV shunting. The onset and progression of BAVMs will be apparent from 5D live brain imaging through cranial windows and will allow for neurobehavioral assessment in these mice. The mouse model of HHT2-BAVM will be useful in assessing HHT2 and in identifying modulators of pathways involved in HHT development, including proteins involved in the Ephrin pathway, the Notch pathway and the Transforming Growth Factor-13 pathway. There are sixteen Erythropoietin-Producing Hepatocellular (Eph) receptors, divided into the A- and B-subclasses: EphA (1-10) and EphB (1-6). They share the same structural features, including an N-terminal extracellular domain that binds with their respective ephrin ligands, a short single-pass hydrophobic transmembrane domain, and an intracellular cytoplasmic signaling domain containing a canonical tyrosine kinase catalytic domain, as well as other protein interaction sites. The ligands for the Eph receptors are the ephrins (also known as Eph-receptor-interacting proteins). Ephrins comprise nine different molecules, also divided into A- and B-subclasses. There are six A-subclass ephrins (ephrin-A1 to ephrin-A6) and three B-subclass ephrins (ephrin-B1 to ephrin-B3). Members of the ephrin A-subclass possess a globular extracellular domain that preferentially only binds EphA receptors and is tethered to the outer leaflet of the plasma membrane by a glycosylphosphatidylinositol linkage. In contrast, the three B-subclass ephrins have an extracellular structure that preferentially only binds to EphB receptors (except for EphA4, which can interact with both A- and B-subclass ephrins). Like the Eph receptors, these B-subclass ephrins possess a single-pass hydrophobic transmembrane domain. However, unlike the Eph receptors, these ligands (commonly referred to as ephrin Bs) do not have an intracellular catalytic domain. Instead, they have a short, highly conserved cytoplasmic tail. They are capable of bidirectional signaling, eliciting both forward as well as reverse signaling.

Also contemplated is a mouse model of HHT2 with one Alk1 germline knockout allele and one floxed Alk1 allele, to provide an alternative genetic basis for assessing HHT2 and for identifying modulators of the aforementioned pathways involved in HHT development, including proteins involved in the Ephrin pathway, the Notch pathway and the Transforming Growth Factor-β pathway.

More particularly, the HHT2-BAVM mouse model (homozygous Alk1 mutation) is expected to identify triggers that lead to BAVM formation, including AV programming, endothelial barrier, inflammation, endothelial-to-mesenchymal transition (EndMT), and superoxide production in mice with Alk1 deletion in the brain endothelium. In addition, ribosomal profiling is contemplated to identify molecular candidates downstream of Alk1 in a genome-wide expression analysis and to obtain global gene expression patterns after Alk1 deletion in the brain endothelium. Using bioinformatic techniques, identified genes are categorized based on their functional characteristics, especially as they relate to processes that may contribute to BAVM formation in the HHT2-BAVM mouse model. Findings are validated by immunofluorescence, qPCR, and/or in situ hybridization by RNA-scope. Identified genes are expected to be molecular regulators and mediators crucial for HHT2 BAVM development.

The HHT2-BAVM mouse model has been used to develop a method to treat HHT, showing that inhibiting EphB4 can attenuate disease progression. Further, it is shown that stimulation of ephrin B2 can have an analogous effect on HHT2. It is expected that inhibition of an Eph type-B receptor, such as EphB1, EphB2, EphB3 or EphB6 in addition to EphB4 will have a beneficial attenuating effect on the progression of Hereditary Hemorrhagic Telangiectasia, including HHT2. If is further expected that stimulating an ephrin type-B ligand polypeptide, including ephrin B1 and ephrin B3 as well as ephrin B2 will have a beneficial effect on the progression of HHT, such as HHT2.

The following examples are presented by way of illustration and are not intended to limit the scope of the subject matter disclosed herein.

### EXAMPLES

### Example 1

Engineering a Mouse Model of HHT2

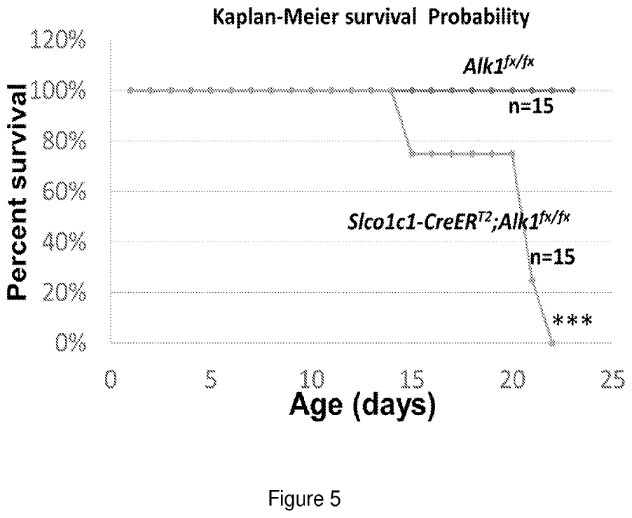

Alk1 was specifically from brain ECs, avoiding the lethal complications of whole-body Alk1 deletion, using the Slco1c1-CreER^{T2} allele, a temporally inducible Cre under the control of the Slco1c1 promoter, which is a promoter active specifically in brain endothelium. Deletion of Alk1 from postnatal day (P) 13 with tamoxifen (TAM) in this Slco1c1-CreER^{T2}; Alk1^{fx/fx} model led to 100% BAVM and intracranial hemorrhages, without detectable defects elsewhere in the body. Although this mouse model has 100% penetrance for BAVM formation, the model is readily adaptable for use with particular chosen time points to compare AVM onset and size, extent of hemorrhage, moribundity time (FIG. 5), and other readouts to ascertain changes in BAVM formation in response to various experimental stimuli. This model of HHT2, and related mouse models constructed, e.g., using the Slco1c1-CreER^{T2} allele are used to test additional triggers in AVM formation, including AV programming, endothelial barrier permeability, endothelial-to-mesenchymal transition (EndMT), inflammation, and superoxide production. In addition ribosomal profiling is used to identify transcriptional targets of Alk1 in brain endothelial cells. Bioinformatic tools are then used to elucidate the functions of encoded gene products.

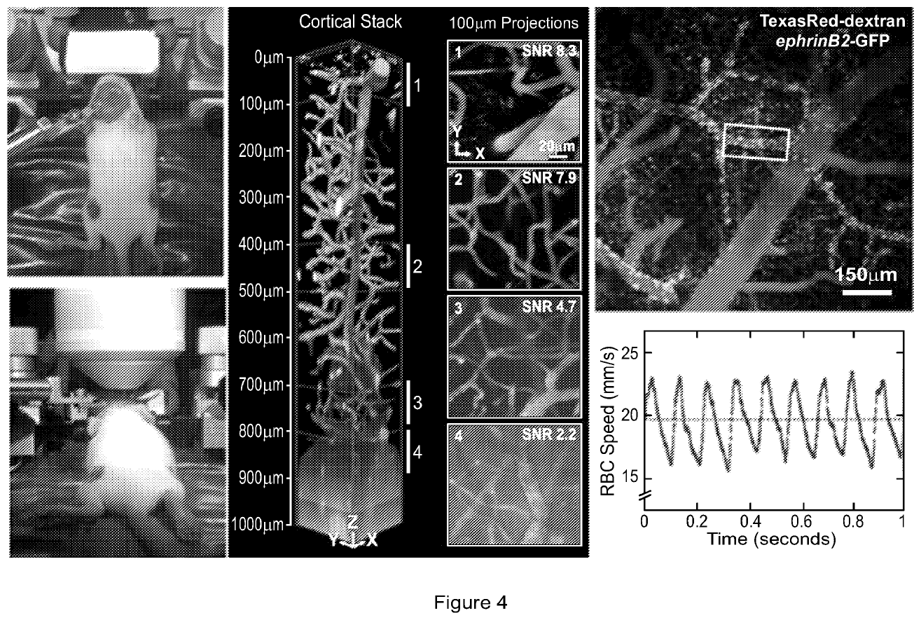

The mouse model of HHT2 is a valuable preclinical tool for understanding pathogenesis, identifying new therapeutic targets, and informing new treatment strategies for HHT2. The model is also useful in testing the hypothesis that abnormal AV programming underlies HHT2 AVM development. We previously characterized AV molecular programming in AVMs, but in a Notch-based model of AVM, rather than an HHT model. The Notch work was predicated on the established premise that Notch regulates AV fate, and we showed that Notch arterializes veins in AVMs. It was our expectation that HHT genes would also affect AV programming in AVM formation, but there was no empirical data supporting this position. The mouse model disclosed herein allows for the testing of this hypothesis by examining AVMs in mice lacking brain endothelial Alk1. The versatile mouse model of HHT2 disclosed herein is also useful in testing whether superoxide production underlies HHT2 AVM formation. These efforts are aided by the in vivo 2-photon (2P) microscopy protocol (FIG. 4) we have developed, which overcomes the barrier of poor accessibility to brain vasculature in live animals. The result of using this technique is 3D imaging with sub-cellular resolution of vascular architecture and blood flow velocities over time (5D), allowing dynamic assessment of AVM formation at cellular resolution. This technique is useful in further elucidating the mechanism underlying BAVM as well as implementing methods of screening for HHT therapeutics among candidate compounds.

The mouse model of HHT is also beneficial in using a lineage tracing approach to track whether EndMT contributes to HHT2 AVM pathogenesis. It is also contemplated that the approach taken in engineering the mouse model of HHT, i.e., the use of the Slco1c1-CreER^{T2} allele to target site-specific mutations in brain ECs, to further our understanding of BAVM. The experimental data disclosed herein also establishes the value of ribosomal profiling to generate an unbiased genome-wide expression profile to identify Alk1 transcriptional targets in brain ECs. The experiments disclosed herein overcome a barrier in the field, establishing a mouse model of HHT2 that reveals molecular triggers in AVM pathogenesis, thus identifying new therapeutic targets.

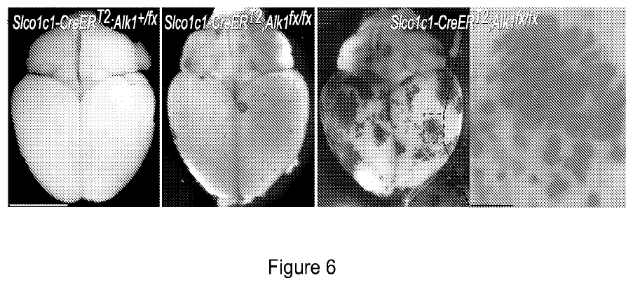

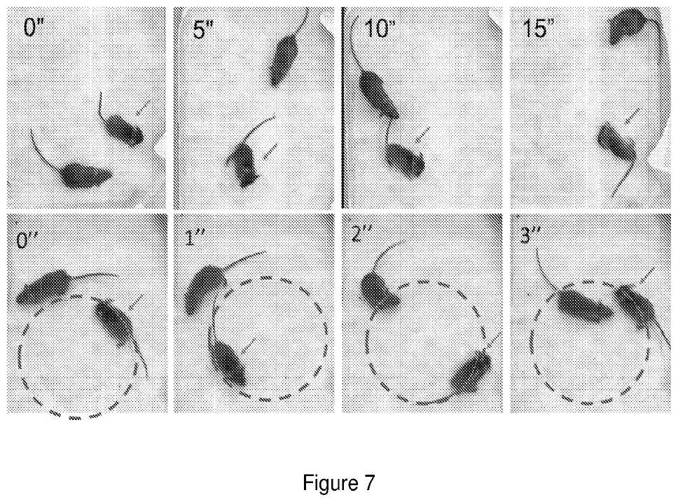

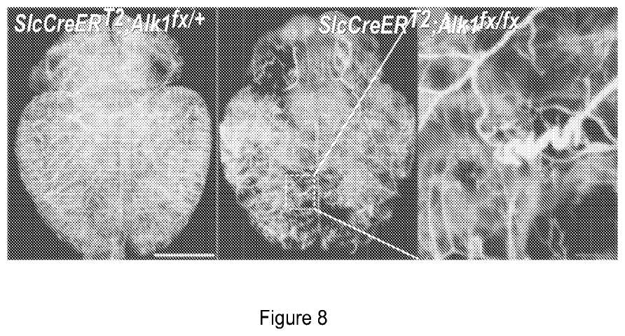

To investigate the function of cerebral endothelial Alk1 in regulating brain vascular structure and function postnatally, Alk1 was specifically deleted in the brain endothelium using a novel mouse genetic tool, i.e., the Slco1c1-CreER^{T2} allele. In the Slco1c1-CreER^{T2}; Alk1^{fx/fx} mouse strain, CreER^{T2} is driven by the brain endothelial specific promoter Slco1c1, which allows deletion of both floxed Alk1 alleles in the brain endothelium. The data disclosed herein shows that deleting both floxed Alk1 alleles from P13 led to HHT-like symptoms by P22 in 100% of mice (FIG. 5), including cerebral hemorrhages (FIG. 6), illness, neurological defects (FIG. 7), and AV shunting (FIG. 8). The longer survival of these mice following disease onset compared to previous models provides an advantage in characterizing molecular changes that lead to BAVM formation.

Mutant and littermate control mice (Table 1) are generated by breeding Slco1c1-CreER^{T2} Alk1^{fx/+} mice with Alk1^{fxf/x} mice. Both parental lines were established prior to breeding. Tamoxifen (TAM; Sigma) (0.5 mg) is injected intraperitoneally (IP) at P13 to delete the floxed Alk1 alleles from the brain endothelium. If needed, the dose and time of TAM injection is optimized to most closely model HHT phenotypes. To verify Alk1 gene deletion, immunostaining is performed using a well-established commercial antibody against mouse Alk1^{24,30} at 2 and 4 days after TAM injection. Deletion of Alk1 in ECs systemically led to defects two days after TAM injection. The following analyses are performed on the mice. A moribundity curve is generated. Moribundity is assessed in an unbiased manner by a daily routine health check by trained veterinary nurses who are unaware of the experimental status of the mice, and through researchers recording animal weight, activity, posture, and appearance. We will monitor neurological behavior is monitored as part of routine observations and video recordings are made of the onset and occurrence of any neurodysfunction, Once moribund, mice are harvested for analysis.

TABLE 1
Brain Endothelial Cell (EC) Alk1 Deletion

Slco1c1-CreER^{T2}; Alk1^{fxfx+}
Slco1c1-CreER^{T2}; Alk1^{fx/+}
Slco1c1-CreER^{T2}; Alk1^{+/+}

Moribund mutant and control mice will be dissected to assess the most severe phenotype, gross pathology, heart/body weight ratio, brain microbleeds, vascular defects, and brain abnormalities. These analyses are also performed at P14 (1 day after TAM, no expected detectable abnormalities), P15 (2 days after TAM, expected detectable abnormalities), and P17 (4 days after TAM, expected intermediate phenotype), to characterize initial defects. Histopathological evaluation and a hypoxia assay with Hypoxyprobe™ immunostaining (HPi-100, HPI, Inc.) is performed using published protocols^{2,33} as AV shunting results in hypoxia.

At P15, P17, and moribund (i.e., the time moribundity occurs), brain vascular structure is evaluated by perfusion with fluorophore-labeled lectin (Vector Labs), alone or with immunostaining for ECs using an anti-CD31 antibody (BD Pharmingen). Mouse parietal cortex is sectioned to 2-3 mm, stained, and flat-mounted to image cortical surface vessels. Densities and patterns of all vessels (CD31+) and perfused vessels (lectin+) are quantified by Image J. EC proliferation is evaluated by Ki67 staining and apoptosis is evaluated by cleaved caspase-3 staining, along with Erg co-staining to label EC nuclei in frozen sections. Five sections per mouse brain are quantified by Image J.

AV shunting is assessed by a microsphere passage assay at P17 and moribund. In this assay, 15 μm FITC-labeled beads, too large to pass through normal brain capillaries, are injected into carotid arteries. If abnormal brain capillaries are present, beads pass through the brain and lodge in the lungs, functionally defining brain AV shunts. Vascular topology is assessed in whole brains by casting with MICROFIL® compound (FlowTech, Inc.) at moribund. We will determine if bleeding occurs before and independently of AVM. Lectin perfusion and gross pathologic analysis will be performed as described herein to detect hemorrhage. Subsequently, half of the brain is stained with H&E, and the other half is used for vascular imaging to detect AVMs.

Experiments are conducted and data is obtained blindly to test group genotypes. Inclusion of mice with different biological variables, including sex, ensures a rigorous comparison in all experiments. Statistical analyses are used to determine the sample size and outcome of all experiments. Differences between two groups are analyzed by Student's t-test. Differences between multiple groups are analyzed by ANOVA, followed by Tukey's post-test for pairwise comparisons. If a normal distribution cannot be assumed, the non-parametric Mann-Whitney U test and Kruskal-Wallis test are used in place of the t-test and ANOVA, respectively. Statistical significance is assumed when p<0.05. For example, to detect a difference of 20 μm in AV connection diameter between groups, assuming a standard deviation of 2.5 μm in controls and 20 μm in mutants, 10 mice are needed per treatment group, based upon a 2-tailed power calculation with power >0.80. Sample sizes for proposed experiments are assessed by power analysis with appropriate parameters.

We expect that the experiments described in this Example will provide a comprehensive characterization of the HHT2 mouse model disclosed herein, documenting the core pathologies and kinetics of their development, including AV shunting, bleeding, behavior changes, and illness.

### Example 2

Second HHT2 Mouse Model that Simulates Human Disease

An HHT2 mouse model is also developed using Slco1c1-CreER^{T2}; Alk1^{-/fx}x, which more closely reflects the dominant genetic lesion of human HHT2. In this mouse model, the null allele represents the germline ALK1 mutation seen in HHT patients. The floxed Alk1 allele is excised in brain ECs, causing loss of heterozygosity (LOH) in these cells. We will characterize the phenotypes in this model are characterized and compared to those in the Slco1c1-CreER^{T2} Alk1^{fx/fx} model.

Mutant and littermate control mice (Table 2) are generated by breeding Slco1c1-CreER^{T2} Alk1^{fx/+} with Alk1^{-/fx} mice. Mice are injected IP with 0.5 mg TAM at P13 to delete the floxed Alk1 allele from brain ECs. Mouse weight, activity, and moribundity are analyzed as described in Example 1. Results are compared to existing data on Slco1c1-CreER^{T2} Alk1^{fxf/x} mice. The TAM regimen is optimized to establish a model with a longer healthy period to better resemble the human disease. With the optimized TAM regimen, gross pathology, histology, and vascular structure are analyzed, as described in Example 1.

TABLE 2
Brain EC Alk1 deletion

Slco1c1-CreER^{T2}; Alk1^{-/fx}
Slco1c1-CreER^{T2}; Alk1^{-/+}
Slco1c1-CreER^{T2}; Alk1^{+/+}

The Slco1c1-CreER^{T2}, Alk1^{-/fx} model is expected to be ideal for modeling human HHT2 and will develop BAVM like the Slco1c1-CreER^{T2}; Alk1^{fx/fx} mice, but BAVM will occur more quickly with the same TAM regimen, as there is only one floxed allele to excise. We expect to identify the optimal TAM regimen to achieve a mouse model most similar to human disease progression.

### Example 3

Initiation and Progression of BAVMs in Slco1c1-CreER^{T2}; Alk1^{fxf/x} Mice Using 5D Two Photon Live Imaging

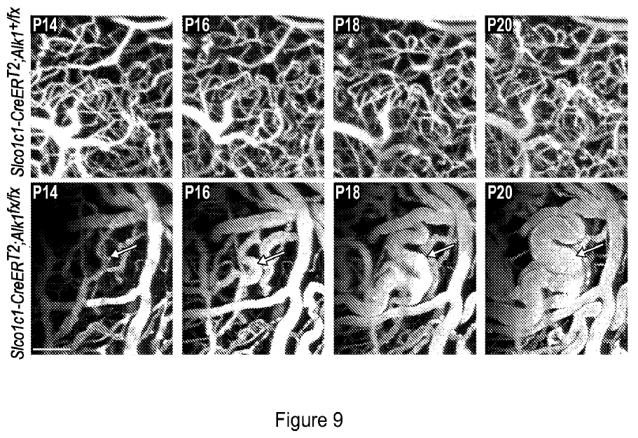

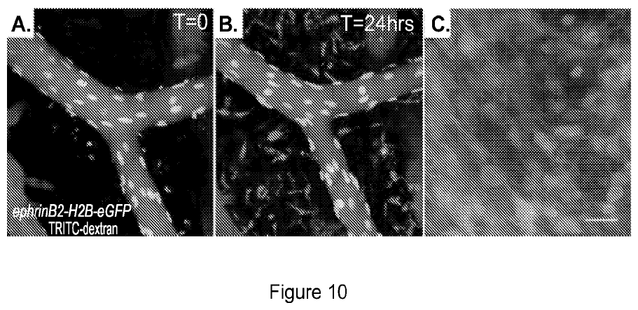

To reveal the development of Alk1-mediated BAVM formation longitudinally, live 5D imaging (FIG. 4) is performed using our custom-built 2-photon microscope. Slco1e1-CreER^{T2}, ALk1^{fxf/x} mice were generated as disclosed herein and examined using time-lapse 2-photon imaging through a cranial window, which showed that the mice developed AV shunts through enlargement of capillary-like vessels, similar to human HHT (FIG. 9). Live brain vasculature is imaged with submicron resolution of vessel diameter and blood velocity, with close to 1000 μm imaging depth in the cortex. ephrin-B2H2B-eGFP (FIG. 10) marks arterial cells and the R26R-RG Cre reporter marks Cre active, i.e., Alk1 deleted, cells^{2,34}. After Cre recombination, the R26R-RG reporter exhibits red nuclear and green cytoplasmic signals (FIG. 10C). These dual reporters allow us for the first time to track ephrin-B2 positive (arterial) and ephrin-B2 negative (non-arterial) ECs in real time to reveal cellular events in AVM formation.

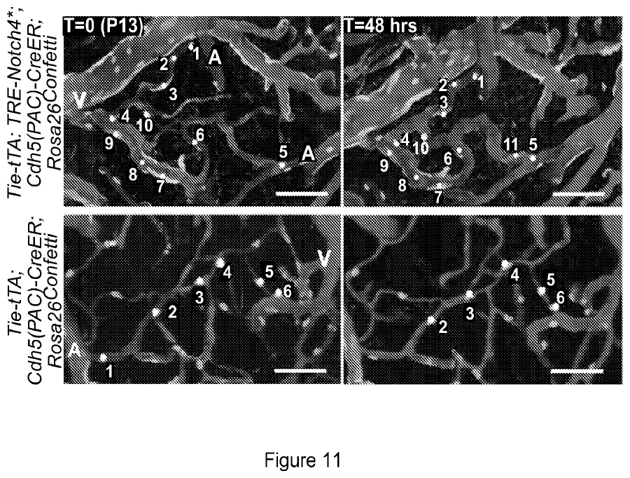

The Notch model of AVM has shown cellular changes leading to AVMs using the Cdh5 (PAC)-CreER^{T2}; R26R-Confetti line², where Cre positive cells have GFP+ nuclei and YFP+ cytoplasm (FIG. 11). Using this marker, each cell and its position is recorded and tracked over time. Specifically, this reporter was used to assess individual cell number (loss or gain) and behavior (migration and the direction), and AV connection diameter. The markers identified herein, i.e., ephrin-B2H2B-eGFP and R26R-RG, represent a technical innovation over the Confetti system, because these markers are able to distinguish arterial versus non arterial (capillary and venous) ECs.

Experimental and control Slco1c1-CreER^{T2}, Alk1^{fxf/x} mice with the arterial nuclear reporter (ephrin-B2H2B-eGFP) and the R26R-RG reporter are generated as in Table 3, by breeding Slco1c1-CreER^{T2}; Alk1^{fx/+}; R26R-RG mice with Alk1^{fxf/x}; ephrin-B2H2BeGFP mice. We have produced the R26R-RG reporter has been constructed. To image the live brain, a cranial window is created over the parietal cortex at P12^{2,34}. Images are taken from P13, prior to TAM injection to induce Alk1 deletion, followed by imaging at P14, P16, P18, and P20 to document the onset and progression of the phenotype^{2,34}. For each imaging session, blood is perfusion-labeled with Cascade Blue-dextran to visualize vessels, allowing for lumen diameter and red blood cell velocity measurements. Through the window, artery, arteriole, capillary, venule, and vein branches are identified by hierarchical structure and blood flow, allowing for determination of the location of marked ECs and the path of migration relative to their vessel compartments^{2,34,36}. The following quantitative data is acquired: AV connection diameter; red blood cell (RBC) velocity in AV connections; and the number, position, and area (“footprint”) of ephrin-B2 positive (arterial) and ephrin-B2 negative (non-arterial) ECs. Changes in: minimal AV connection diameter; RBC velocity; cell number; cell position (rate and direction of migration, rate of directional migration); and cell area are then determined by extrapolation.

TABLE 3
Alk1 deletion with reporters for imaging

Slco1c1-CreER^{T2}; Alk1^{fx/fx}; ephrin-B2_{H2B-eGFP}; R26R-RG
Slco1c1-CreER^{T2}; Alk1^{fx/+}; ephrin-B2_{H2B-eGFP}; R26R-RG
Slco1c1-CreER^{T2}; Alk1^{+/+}; ephrin-B2_{H2B-eGFP}; R26R-RG

It is expected that data on the onset and progression of AVMs in mice lacking Alk1 specifically in brain endothelium, is acquired, providing the first longitudinal, high resolution imaging of HHT2 BAVM development. The time of AVM initiation also provides crucial information as to whether bleeding occurs prior to AVM formation.

### Example 4

Mechanisms of BAVM Development in Mice with Brain-Endothelial-Specific Alk1 Deletion

The mouse model disclosed herein is used to investigate candidate events leading to BAVM progression in HHT2 including AV programming, inflammation, endothelial barrier, EndMT, and superoxide production. Although the mouse model has 100% penetrance of BAVMs, carefully chosen time points are used to compare AVM onset and size, extent of hemorrhage, moribundity time, and other readouts to ascertain effects of a candidate trigger.

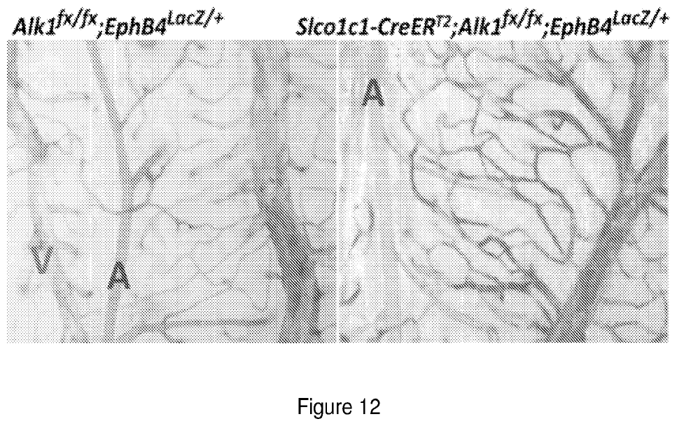

An avenue of inquiry relevant to BAVM development in mice with brain-specific Alk1 deletions is the role of AV molecular programming, which is investigated using Slco1c1-CreER^{T2}; Alk1^{fx/fx} mice. We have characterized AV molecular programming in AVMs in a Notch-based model of AVM. The Notch work was predicated on the established premise that Notch regulates AV fate, and we showed that Notch arterializes veins in AVMs. Inspired by the Notch work, in which we showed that AV specification/programming is a key molecular mechanism in AVM formation, we expected that HHT genes would also affect AV programming in AVM formation. There is evidence that Alk1 knockout animals have reduced ephrinB2 expression in embryos³⁷. Without wishing to be bound by theory, we expect Alk1 loss of function to venulize arteries. The experiments disclosed herein will assess this expectation in the HHT2 AVM setting. Data from experiments performed to date show that EphB4 expression was not changed in arteries lacking Alk1, rather EphB4 expression expanded into capillaries in this background (FIG. 12).

### Example 5

LacZ Reporter Assays Identify the Molecular Identities of Alk1 Mutant Vessels

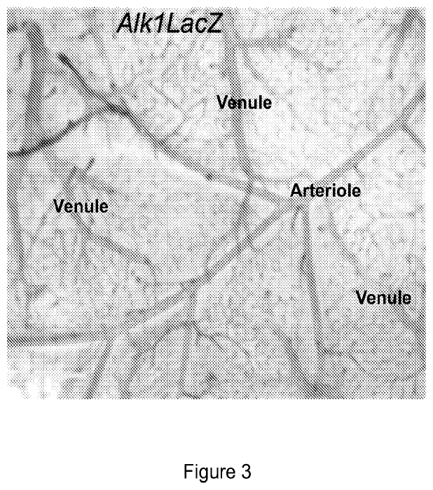

AVMs are a nidus of enlarged vessels connected by abnormal AV shunting. AVMs have historically been investigated as abnormal vessel growth, which may be a consequence of other primary lesions. We have proposed that abnormal AV programming underlies AVM development in a Notch AVM mouse model, where we showed that Notch arterialized veins^{2,34}. Based on the data that Alk1 is primarily expressed in arteries and not in veins²⁹ (FIG. 3), we expected Alk1 deletion to disrupt AV programming, leading to AVM formation.

To test the AV molecular identities of Alk1 mutant vessels, LacZ reporter assays^{33,34} are used, where ephrinB2^{LacZ/+} and EphB4^{LacZ4/+} mark arterial and venous vessels, respectively. First, the mice identified in Table 4 are generated by breeding Slco1c1-CreER^{T2}; Alk1^{fx/+} mice with Alk1^{fx/fx}; ephrinB2^{LacZ/+} or Alk1^{fx/fx}; EphB4^{LacZ/+} mice. The mice identified by asterisks in Table 4 are analyzed by LacZ staining at P16, following TAM injection at P13 (FIG. 12). AV identities are confirmed by immunostaining for Cx40 (arterial marker; Santa Cruz Biotechnology) or CoupTFII (venous marker; R&D Systems) in mice shown in Table 1 (without LacZ alleles), at P16 following TAM injection at P13.

TABLE 4
Alk1 deletion with AV reporters

*Slco1c1-CreER^{T2}; Alk1^{fx/fx}; ephrinB2^{LacZ/+}
*Slco1c1-CreER^{T2}; Alk1^{fx/+}; ephrinB2^{LacZ/+}
Slco1c1-CreER^{T2}; Alk1^{fx/fx}; ephrinB2^{LacZ/+}
Slco1c1-CreER^{T2}; Alk1^{fx/fx}
*Slco1c1-CreER^{T2}; Alk1^{fx/fx}; EphB4^{LacZ/+}
*Slco1c1-CreER^{T2}; Alk1^{fx/+}; EphB4^{LacZ/+}
Slco1c1-CreER^{T2}; Alk1^{fx/fx}; EphB4^{LacZ/+}
Slco1c1-CreER^{T2}; Alk1^{fx/fx}

We originally expected reduced arterial markers and increased venous markers in arteries in Alk1-deficient mice. However, the data show no change in these markers in arteries, but upregulation of EphB4 in veins and capillaries as revealed by LacZ staining in Slco1c1-CreER^{T2} Alk1^{fx/fx}; EphB4^{LacZ/+} mice following TAM treatment at P13 (FIG. 12). This finding is consistent with our expectation in terms of the increase in EphB4 expression. Immunostaining of the EphB4 protein in Alk1 mutant brain tissue is also performed.

### Example 6

Determining Whether ephrinB2 or EphB4 is Required for BAVM Formation in Alk1 Mutant Mice

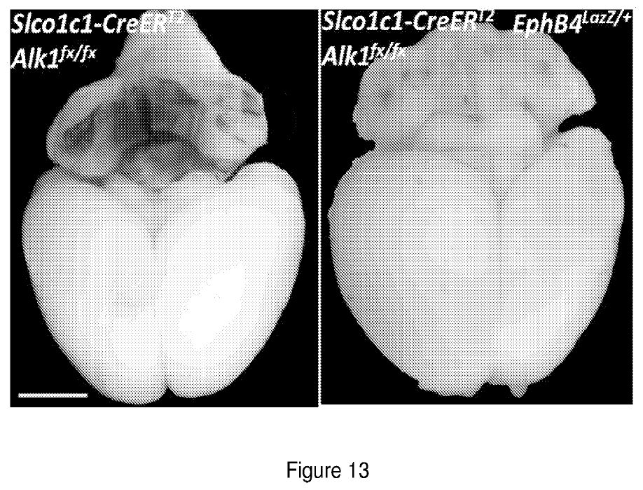

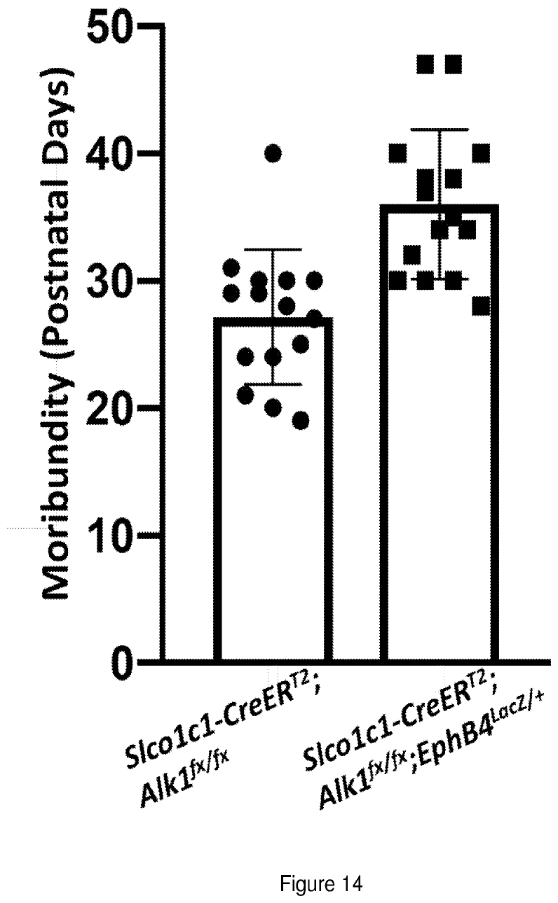

The LacZ reporters described herein are knockins (i.e., one copy of ephrinB2 or EphB4 is knocked out by the LacZgene). Experiments will reveal whether having heterozygous ephrinB2 or EphB4 affects the BAVM phenotype in mice lacking Alk1. The data show that Slco1c1-CreER^{T2}; Alk1^{fx/fx}; EphB^{LacZ/+} mice treated with TAM from P13 show reduced BAVM formation and hemorrhage and delayed moribundity compared to Slco1c1-CreER^{T2}; Alk1^{fx/fx} mice (FIGS. 13 and 14).

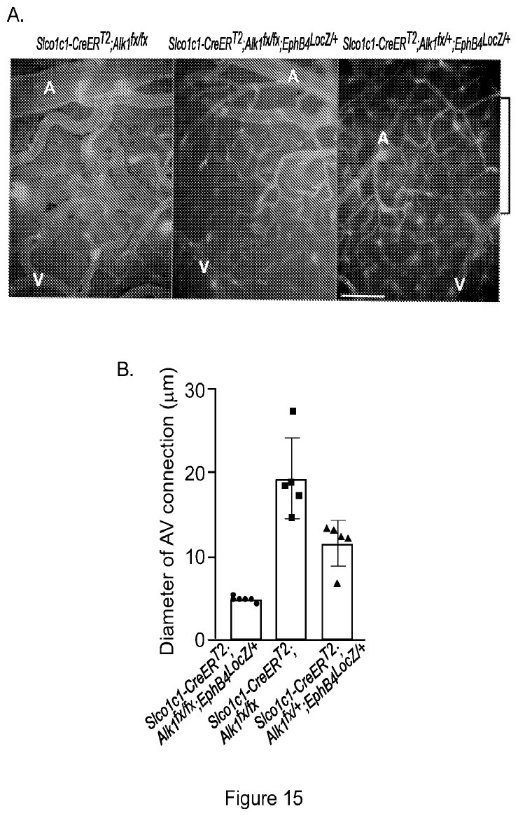

To determine if having heterozygous ephrinB2 or EphB4 affects the BAVM phenotype in mice lacking Alk1, the mice identified in Table 4 in black letters are analyzed using methods described herein. For the EphB4 study, BAVM formation has been examined at the single time point of P20 (FIG. 15). Also, BAVM formation is examined in Slco1c1-CreER^{T2}; Alk1^{fx/fx}; EphB4^{LacZ/+} and control mice over time through a cranial window, as described herein. Blood is perfusion-labeled with Cascade Bluedextran to visualize vessels and allow for lumen diameter and red blood cell velocity measurements. Whether AVM formation is delayed is also assessed in these mice. Analogous experiments are performed in the Slco1c1-CreER^{T2}; Alk1^{fx/fx}; ephrinB2^{LacZ/+} line.

A pharmacological approach to inhibition of EphB4 is also undertaken. The soluble extracellular domain of EphB4 (sEphB4) completely inhibits EphB4 signaling in mice³⁸. sEphB4 is injected intraorbitally³⁴ into Slco1c1-CreER^{T2}; Alk1^{fx/fx} mice to test its ability to prevent and treat BAVMs. Imaging studies through a cranial window offers unprecedented insight into the efficacy of a candidate drug such as sEphB4 in AVM prevention and regression in real time. Such a study without a cranial window would require a great number of experimental mice and would lack definitive proof that an established AVM had regressed. To determine if sEphB4 prevents BAVM formation, at P12 a cranial window is implanted and also begin daily injections of sEphB4 (about 4 mg/kg) into Slco1c1-CreERT2; Alk1fx/fx mice are begun. Imaging begins at P13, followed by immediate TAM injection and imaging at P14, P16, P18, and P20. To determine if sEphB4 causes regression of BAVMs, a second cohort of mice are treated with TAM at P13, followed by cranial window implantation at P17. Mice are imaged at P18, after BAVM formation, followed by sEphB4 treatment daily, and imaging at P19, 20, and 22. Recombinant human fibronectin is injected as a negative control. We expect the experiment to reveal that pharmacological repression of EphB4, like genetic reduction of EphB4, leads to prevention or reduction of BAVMs in Alk1 mutant mice. More generally, we expect ephrinB2 and EphB4 to be important for Alk1-mediated AVM formation, and we expect that heterozygosity of these genes (i.e., mice harboring heterozygous mutant Alk1 in brain ECs) will affect AVM formation, as revealed in our Slco1c1-CreER^{T2} Alk1″ model. Data from completed experiments show that heterozygous deletion of EphB4 inhibits cerebral hemorrhage and delays moribundity (FIGS. 13, 14) and reduces AV shunting (FIG. 15) following Alk1 deletion in the mouse model disclosed herein, compared to control mice. These findings are highly significant and have a high impact for future potential treatment of BAVM. In addition, the success of this experiment shows the feasibility of the general approach disclosed herein. That general approach involves a genetic approach to understanding the efficacy of gene deletion or reduction in reducing a phenotype (in this case BAVM), and then taking a pharmacological approach to reducing the same gene. This general approach can also be applied to the other genes in the ephrin pathway as disclosed herein that are expected to play a role, or be capable of playing a role, in promoting BAVM formation. In addition, the disclosure contemplates an expectation that genes identified as exhibiting altered expression, e.g., by ribosomal profiling in mice containing Alk1 mutations in brain ECs relative to expression in wild-type mice will be genes involved in BAVM formation. Screens for compounds altering the expression, or activity of the encoded gene product, of Alk1, other genes in the ephrin pathway, and genes identified by the above-described ribosomal profiling in Alk1 mice, are contemplated by the disclosure.

### REFERENCES

1 Ruiz-Llorente, L. et al. Endoglin and alk1 as therapeutic targets for hereditary hemorrhagic telangiectasia. Expert opinion on therapeutic targets 21, 933-947 (2017).
2 Murphy, P. A. et al. Constitutively active Notch4 receptor elicits brain arteriovenous malformations through enlargement of capillary-like vessels. Proc Natl Acad Sci USA 111, 18007-18012 (2014).
3 Zhou, P. et al. Interrogating translational efficiency and lineage-specific transcriptomes using ribosome affinity purification. Proc Natl Acad Sci USA 110, 15395-15400 (2013).
4 Friedlander, R. M. Clinical practice. Arteriovenous malformations of the brain. N Engl J Med 356, 2704-2712 (2007).
5 Young, W. L. & Yang, G. Y. Are there genetic influences on sporadic brain arteriovenous malformations? Stroke 35, 2740-2745 (2004).
6 Guttmacher, A. E., Marchuk, D. A. & White, R. I., Jr. Hereditary hemorrhagic telangiectasia. N Engl J Med 333, 918-924 (1995).
7 Shovlin, C. L. et al. Diagnostic criteria for hereditary hemorrhagic telangiectasia (Rendu-Osler-Weber syndrome). Am J Med Genet 91, 66-67 (2000).
8 Finsterer, J. & Sehnal, E. Hypercoagulability in hereditary hemorrhagic telangiectasia with epilepsy. Journal of neurosciences in rural practice 6, 407-409 (2015).
9 Brinjikji, W., Iyer, V. N., Sorenson, T. & Lanzino, G. Cerebrovascular Manifestations of Hereditary Hemorrhagic Telangiectasia. Stroke 46, 3329-3337 (2015).
10 Morgan, T. et al. Intracranial hemorrhage in infants and children with hereditary hemorrhagic telangiectasia (Osler-Weber-Rendu syndrome). Pediatrics 109, E12 (2002).
11 Hereditary hemorrhagic telangiectasia, https://ghr.nlm.nih.gov/condition/hereditary-hemorrhagictelangiectasia# (2016).
12 Hereditary Hemorrhagic Telangiectasia, https://rarediseases.org/rare-diseases/hereditary-hemorrhagictelangiectasia/ (2017).
13 Abdalla, S. A. & Letarte, M. Hereditary haemorrhagic telangiectasia: current views on genetics and mechanisms of disease. J Med Genet 43, 97-110 (2006).
14 Johnson, D. W. et al. Mutations in the activin receptor-like kinase 1 gene in hereditary haemorrhagic telangiectasia type 2. Nat Genet 13, 189-195 (1996).
15 McDonald, J Bayrak-Toydemir, P. & Pyeritz, R. E. Hereditary hemorrhagic telangiectasia: an overview of diagnosis, management, and pathogenesis. Genet Med 13, 607-616 (2011).
16 Vincent, P. et al. A third locus for hereditary haemorrhagic telangiectasia maps to chromosome 12q. Hum Mol Genet 4, 945-949 (1995).
17 Johnson, D. W. et al. A second locus for hereditary hemorrhagic telangiectasia maps to chromosome 12. Genome Res 5, 21-28 (1995).
18 Berg, J. N. et al. The activin receptor-like kinase 1 gene: genomic structure and mutations in hereditary hemorrhagic telangiectasia type 2. Am J Hum Genet 61, 60-67 (1997).
19 ten Dijke, P. & Hill, C. S. New insights into TGF-beta-Smad signalling. Trends Biochem Sci 29, 265-273 (2004).
20 Kim, H. et al. Genetic considerations relevant to intracranial hemorrhage and brain arteriovenous malformations. Acta Neurochir Suppl 105, 199-206 (2008).
21 Tual-Chalot, S., Oh, S. P. & Arthur, H. M. Mouse models of hereditary hemorrhagic telangiectasia: recent advances and future challenges. Front Genet 6, 25 (2015).
22 Gkatzis, K. et al. Interaction Between ALK1 Signaling and Connexin40 in the Development of Arteriovenous Malformations. Arterioscler Thromb Vasc Biol 36, 707-717 (2016).
23 Tual-Chalot, S. et al. Endothelial Depletion of Acvrll in Mice Leads to Arteriovenous Malformations Associated with Reduced Endoglin Expression. PLOS ONE 9, e98646 (2014).
24 Lee, H. W. et al. Alk2/ACVR1 and Alk3/BMPR1A Provide Essential Function for Bone Morphogenetic Protein-Induced Retinal Angiogenesis. Arterioscler Thromb Vasc Biol 37, 657-663 (2017).
25 Nielsen, C. M., Huang, L., Murphy, P. A., Lawton, M. T. & Wang, R. A. Mouse models of cerebral arteriovenous malformation. Stroke 47, 293-300 (2016).
26 Mohr, J. P. et al. Medical management with or without interventional therapy for unruptured brain arteriovenous malformations (ARUBA): a multicentre, non-blinded, randomised trial. Lancet 383, 614-621 (2014).
27 Attisano, L. et al. Identification of human activin and TGF beta type I receptors that form heteromeric kinase complexes with type II receptors. Cell 75, 671-680 (1993).
28 ten Dijke, P. et al. Activin receptor-like kinases: a novel subclass of cell-surface receptors with predicted serine/threonine kinase activity. Oncogene 8, 2879-2887 (1993).
29 Roman, B. L. & Hinck, A. P. ALK1 signaling in development and disease: new paradigms. Cell Mol Life Sci 74, 4539-4560 (2017).
30 Ola, R. et al. P13 kinase inhibition improves vascular malformations in mouse models of hereditary haemorrhagic telangiectasia. Nature Communications 7, 13650 (2016).
31 Lang, M. F. et al. A transgenic approach to identify thyroxine transporter-expressing structures in brain development. J Neuroendocrinol 23, 1194-1203 (2011).
32 Ingolia, N. T., Ghaemmaghami, S., Newman, J. R. S. & Weissman, J. S. Genome-Wide Analysis in Vivo of Translation with Nucleotide Resolution Using Ribosome Profiling. Science 324, 218 (2009).
33 Nielsen, C. M. et al. Deletion of Rbpj from postnatal endothelium leads to abnormal arteriovenous shunting in mice. Development 141, 3782-3792 (2014).
34 Murphy, P. A. et al. Notch4 normalization reduces blood vessel size in arteriovenous malformations. Sci Transl Med 4, 117ra118 (2012).
35 Shioi, G. et al. A mouse reporter line to conditionally mark nuclei and cell membranes for in vivo liveimaging. Genesis 49, 570-578 (2011).
36 Kim, T. N. et al. Line-scanning particle image velocimetry: an optical approach for quantifying a wide range of blood flow speeds in live animals. PloS one 7, e38590 (2012).
37 Urness, L. D., Sorensen, L. K. & Li, D. Y. Arteriovenous malformations in mice lacking activin receptor-like kinase-1. Nat Genet 26, 328-331, doi:10.1038/81634 (2000).
38 Kertesz, N. et al. The soluble extracellular domain of EphB4 (sEphB4) antagonizes EphB4-EphrinB2 interaction, modulates angiogenesis, and inhibits tumor growth. Blood 107, 2330-2338 (2006).
39 Chen, Y. et al. Interleukin-6 involvement in brain arteriovenous malformations. Ann Neurol 59, 72-80 (2006).
40 Chen, Y. et al. Evidence of inflammatory cell involvement in brain arteriovenous malformations. Neurosurgery 62, 1340-1349; discussion 1349-1350 (2008).
41 Storer, K. P., Tu, J., Karunanayaka, A., Morgan, M. K. & Stoodley, M. A. Inflammatory molecule expression in cerebral arteriovenous malformations. J Clin Neurosci 15, 179-184 (2008).
42 Mitrofan, C. G. et al. Bone morphogenetic protein 9 (BMP9) and BMP10 enhance tumor necrosis factor-alpha-induced monocyte recruitment to the vascular endothelium mainly via activin receptor-like kinase 2. (2017).
43 Zhang, R. et al. Persistent infiltration and pro-inflammatory differentiation of monocytes cause unresolved inflammation in brain arteriovenous malformation. Angiogenesis 19, 451-461 (2016).
44 Chen, W. et al. Reduced mural cell coverage and impaired vessel integrity after angiogenic stimulation in the Alk1-deficient brain. Arterioscler Thromb Vasc Biol 33, 305-310 (2013).
45 Aoki, T. et al. PGE(2)-EP(2) signalling in endothelium is activated by haemodynamic stress and induces cerebral aneurysm through an amplifying loop via NF-kappaB. British journal of pharmacology 163, 1237-1249 (2011).
46 Johnston, W. F. et al. Inhibition of interleukin-1beta decreases aneurysm formation and progression in a novel model of thoracic aortic aneurysms. Circulation 130, S51-59 (2014).
47 ljaz, T. et al. Deletion of NF-kappaB/RelA in Angiotensin II-Sensitive Mesenchymal Cells Blocks Aortic Vascular Inflammation and Abdominal Aortic Aneurysm Formation. Arterioscler Thromb Vasc Biol 37, 1881-1890 (2017).
48 Gomez, D. et al. Interleukin-1beta has atheroprotective effects in advanced atherosclerotic lesions of mice. Nat Med, (2018).
49 Starke, R. M. et al. Critical role of TNF-alpha in cerebral aneurysm formation and progression to rupture. Journal of neuroinflammation 11, 77 (2014).
50 Dave, J. M., Mirabella, T., Weatherbee, S. D. & Greif, D. M. Pericyte ALK5/TIMP3 Axis Contributes to Endothelial Morphogenesis in the Developing Brain. Dev Cell 44, 665-678 e666 (2018).
51 Srinivasan, S. et al. A mouse model for hereditary hemorrhagic telangiectasia (HHT) type 2. Hum Mol Genet 12, 473-482 (2003).
52 Daneman, R., Zhou, L., Kebede, A. A. & Barres, B. A. Pericytes are required for blood-brain barrier integrity during embryogenesis. Nature 468, 562-566 (2010).
53 Vanlandewijck, M. et al. A molecular atlas of cell types and zonation in the brain vasculature. Nature 554, 475-480 (2018).
54 Jung, B., Arnold, T. D., Raschperger, E., Gaengel, K. & Betsholtz, C. Visualization of vascular mural cells in developing brain using genetically labeled transgenic reporter mice. J Cereb Blood Flow Metab 38, 456-468 (2018).
55 Kovacic, J. C. et al. Endothelial to Mesenchymal Transition in Cardiovascular Disease: JACC State-ofthe-Art Review. Journal of the American College of Cardiology 73, 190-209 (2019).
56 Dejana, E., Hirschi, K. K. & Simons, M. The molecular basis of endothelial cell plasticity. Nat Commun 8, 14361 (2017).
57 Jerkic, M., Sotov, V. & Letarte, M. Oxidative stress contributes to endothelial dysfunction in mouse models of hereditary hemorrhagic telangiectasia. Oxid Med Cell Longev 2012, 686972 (2012).
58 Jerkic, M. et al. Pulmonary hypertension in adult Alk1 heterozygous mice due to oxidative stress. Cardiovascular Research 92, 375-384 (2011).
59 Han, B. H. et al. Contribution of reactive oxygen species to cerebral amyloid angiopathy, vasomotor dysfunction, and microhemorrhage in aged Tg2576 mice. Proc Natl Acad Sci USA 112, E881-890 (2015).

All publications and patents mentioned in the application are herein incorporated by reference in their entireties or in relevant part, as would be apparent from context. Various modifications and variations of the disclosed subject matter will be apparent to those skilled in the art without departing from the scope and spirit of the disclosure. Although the disclosure has been described in connection with specific embodiments, it should be understood that the invention as claimed should not be unduly limited to such specific embodiments. Various modifications of the described modes for making or using the disclosed subject matter that are obvious to those skilled in the relevant field(s) are intended to be within the scope of the following claims.

What is claimed is:

1. A method of treating arteriovenous malformation in a subject comprising administering a therapeutically effective amount of a modulator of Eph receptor/ephrinB2 signaling to the subject.

2. The method of claim 1 wherein the modulator inhibits Eph receptor/ephrin B2 signaling.

3. The method of claim 1 wherein the modulator stimulates Eph receptor/ephrin B2 signaling.

4. The method of claim 1 wherein the subject has hereditary hemorrhagic telangiectasia.

5. The method of claim 1 wherein the subject is at risk of, or has had, a hemorrhagic stroke.

6. The method of claim 1 wherein the arteriovenous malformation is in the brain.

7. A method of treating hereditary hemorrhagic telangiectasia in a subject comprising administering a therapeutically effective amount of a modulator of an Eph receptor polypeptide to the subject.

8. The method of claim 7 wherein the modulator is an inhibitor of the Eph receptor.

9. The method of claim 8 wherein the Eph receptor is an Eph type-B receptor.

10. The method of claim 9 wherein the Eph type-B receptor is Eph type-B receptor 4 (EphB4).

11. The method of claim 10 wherein the inhibitor is soluble Eph type-B receptor 4.

12. A method of treating hereditary hemorrhagic telangiectasia in a subject comprising administering a therapeutically effective amount of a modulator of an ephrin polypeptide to the subject.

13. The method of claim 12 wherein the modulator is a stimulator of the ephrin polypeptide.

14. The method of claim 13 wherein the ephrin polypeptide is an ephrin type-B polypeptide.

15. The method of claim 14 wherein the ephrin type-B polypeptide is ephrin B2.

16. A method of screening for a therapeutic to treat hereditary hemorrhagic telangiectasia comprising
(a) administering a candidate compound to an organism with a brain-specific arteriovenous malformation (BAVM organism);
(b) maintaining the organism for a time suitable for AVM symptoms to arise;
(c) measuring a property associated with brain arteriovenous malformation (BAVM) in a BAVM organism; and
(d) comparing the level of the property in the BAVM organism receiving the candidate compound to the level of the property in a BAVM organism not receiving the candidate compound,
wherein a candidate compound is identified as a therapeutic to treat hereditary hemorrhagic telangiectasia if the level of the property differs in the BAVM organism receiving the candidate compound relative to the level of the property in the BAVM organism not receiving the candidate compound.

17. The method of claim 16 wherein the BAVM organism is a mouse.

18. The method of claim 16 wherein the property is a symptom of BAVM.

19. The method of claim 18 wherein the symptom is intracranial bleeding.

20. The method of claim 18 wherein the symptom is hemorrhagic stroke.

21. The method of claim 16 wherein the property is AVM onset, AVM size, extent of hemorrhage, or time to moribundity.

22. An in vitro method of screening for a therapeutic to treat hereditary hemorrhagic telangiectasia comprising
(a) administering a candidate compound to an alk1^{-/-} endothelial cell derived from a brain;
(b) measuring the level of an arteriovenous programming protein; and
(c) comparing the level of the arteriovenous programming protein in the presence of the candidate compound to the level of the arteriovenous programming protein in the absence of the compound,
wherein a candidate compound is identified as a therapeutic to treat hereditary hemorrhagic telangiectasia if the level of an arteriovenous programming protein is differs in the presence of the candidate compound compared to the level in the absence of the candidate compound.

23. The method of claim 22 wherein the arteriovenous programming protein is a protein in the Ephrin pathway.

24. The method of claim 23 wherein the protein in the Ephrin pathway is an ephrin type-B or an Eph type-B receptor.

25. The method of claim 24 wherein the protein in the Ephrin pathway is Eph type-B receptor 4 or ephrin B2.

26. The method of claim 22 wherein the arteriovenous programming protein is a protein in the Notch pathway or the Transforming Growth Factor-β pathway.

27. A non-human mammal comprising a homozygous Alk1⁻ inactivating mutation exclusively in brain endothelial cells.

28. The mammal of claim 27 wherein the mammal is a mouse.

29. The mammal of claim 27 wherein the homozygous Mkt inactivating mutation is a deletion of A/k 1.

30. A method of making the non-human mammal of claim 21 comprising the use of Crispr/Cas9 to introduce the mutation.

31. The method of claim 30 further comprising determining that a brain endothelial cell harbors the mutation by single-cell sequencing.
